(19) United States
(12) Patent Application Publication
(10) Pub. No.: US 2013/0150556 A1
(43) Pub. Date: Jun. 13, 2013

(54) DEAMIDATED BARLEY PROTEINS

(71) Applicant: Lingyun Chen, Edmonton (CA)

(72) Inventor: Lingyun Chen, Edmonton (CA)

(73) Assignee: THE GOVERNORS OF THE UNIVERSITY OF ALBERTA, Edmonton, AB (CA)

(21) Appl. No.: 13/810,473

(22) PCT Filed: Jul. 18, 2011
(86) PCT No.: PCT/CA2011/000831

§ 371 Date: Feb. 21, 2013

Related U.S. Application Data
(60) Provisional application No. 61/364,998, filed on Jul. 16, 2010.
(60) Provisional application No. 61/365,024, filed on Jul. 16, 2010.

(51) Int. Cl. C07K 14/415

(52) U.S. Cl. CPC C07K 14/415
USPC 530/372

(57) ABSTRACT

The invention is directed to a method of producing deamidated barley proteins by extracting protein from barley using an alcohol or alkaline; treating the extracted protein with an alkaline or acidic solution for a sufficient time and temperature for partial deamidation; and recovering partially deamidated protein from the solution. The partially deamidated protein has improved functional properties with respect to solubility, and emulsifying and foaming properties at acidic and neutral pHs compared to those of a non-deamidated protein.

### FIELD OF THE INVENTION

The present invention is directed to deamidated barley proteins.

### BACKGROUND OF THE INVENTION

Deamidation is a chemical modification known to improve solubility and other functional properties of some food proteins (Malabat et al., 2001). In cereal proteins, deamidation is a particularly important modification since up to one-third of their total amino acid content is glutamine (Hamada, 1992; Lan et al., 2010). The conversion of the amide groups on glutamine side chains into acid groups is believed to improve cereal protein solubility as a high content of glutamine residues may cause the aggregation of the protein molecules via hydrogen bonding. Deamidation may also partially unfold the protein and indirectly lead to protein hydrolysis by cleavage of the peptide bond (Cabra et al., 2007). These changes improve the functional properties of wheat, corn, rice and soy proteins, making them useful for the food and pharmaceutical industries (Cabra et al., 2007; Chan and Ma, 1999; Hamada and Marshall, 1989; Li et al., 2009; Li et al., 2010; Matsudomi et al., 1985; Paraman et al., 2007; Yong et al., 2006). However, excessive molecular charges on proteins and peptide bond cleavage could affect the protein structure and cause undesirable functional properties that reduce their utility (Cabra et al., 2007).

In general, systematic research of protein molecular structure and subsequent functionality (as a function of degree of deamidation values) is limited. More specifically, information about protein structure transition at low deamidation levels and subsequent functionality is lacking.

As the fourth most widely cultivated cereal in the world after wheat, rice and corn, barley is gaining increasing popularity as a part of the human diet because of the recent health claim made about its β-glucan (FDA, 2005; Yalcin et al., 2008). This soluble dietary fibre component of barley is known to reduce both blood cholesterol and the glycemic index (Kalra and Jood, 2000; Wood, 2004). Additionally, barley represents a potential abundant and affordable source of plant proteins. The overall barley grain protein content is 8-13% (w/w) depending on the variety (Pomeranz and Shands, 1974).

Glutelin comprises approximately 35-40% of the total barley grain protein, and is one of the main storage proteins of barley (Shewry, 1993). Glutelin exhibits high surface hydrophobicity which markedly reduces protein solubility, hindering glutelin applications.

Hordein is a barley prolamin and comprises approximately 35-55% of the total barley grain protein, and is the main storage protein for barley (Shewry, 1993). Barley hordeins are divided into four groups based on their electrophoretic mobilities and amino acid compositions: the B (30-50 kDa, sulfur-rich) and C (55-80 kDa, sulfur-poor) hordeins (70-80% and 10-20% of the hordein fraction, respectively) and the D (80-90 kDa) and A (15 kDa) hordeins (less than 5% of the total hordein fraction). The A hordeins are likely alcohol-soluble albumins or globulins, or breakdown products of larger hordeins rather than true hordeins. C and some B hordeins appear as monomers, while most B and D hordeins are linked by inter-chain disulfide bridges (Celus et al., 2006).

Hordein is rich in hydrophobic amino acids (40%), with the highest levels corresponding to proline, leucine, and valine (Wang et al., 2010). This amino acid profile results in high protein surface hydrophobicity, which favors rapid adsorption at the hydrophobic interface and then forms a viscoelastic film to stabilize foams and emulsions (Wang et al., 2010). However, these features also result in a marked reduction in water solubility and a tendency of protein aggregation. Both of these changes hinder their functional application, since protein water solubility is critical to impart other desired and necessary properties such as emulsifying and foaming functionalities (Kinsella, 1976).

Barley endosperm proteins such as glutelin and hordein are typically regarded as contaminants by the brewing industry and are precipitated out in the spent grains for use as animal feed. Development of extraction and fractionation techniques of barley proteins and the subsequent characterization of their functional properties may facilitate the diversified opportunities for barley protein fractions in food and non-food applications, and identify value-added applications for barley proteins.

Therefore, there is a need in the art for converting barley byproducts into useful products.

### SUMMARY OF THE INVENTION

The present invention relates to partially deamidated barley proteins having various degrees of deamidation. In one embodiment, the deamidated proteins have improved functional properties with respect to solubility, and emulsifying and foaming at acidic and neutral pHs, compared to those of a non-deamidated protein.

In one aspect, the invention comprises a method of producing a deamidated protein from barley, comprising the steps of:

a) extracting protein from barley;

b) treating the extracted protein with an alkaline or acidic solution for a sufficient time and temperature for partial deamidation; and

c) recovering the partially deamidated protein from the solution.

In one embodiment, the deamidated protein has a degree of deamidation less than about 40%, and preferably less than about 20%, more preferably less than about 10%. In one embodiment, the deamidated protein has a degree of deamidation of between about 0.5% and 5.0%.

In one embodiment, the deamidated protein is glutelin. In one embodiment, in step (a), barley endosperm flour is mixed with an alkaline solution adjusted with sodium hydroxide. In one embodiment, the ratio of the solution to flour is 10:1 (v/w). In one embodiment, the mixture is stirred for about half an hour at room temperature. In one embodiment, glutelin is precipitated and freeze-dried.

In one embodiment, the deamidated protein is hordein. In one embodiment, in step (a), pearled grain flour is mixed with ethanol. In one embodiment, the ratio of ethanol to flour is 6:1 (v/w). In one embodiment, the mixture is stirred for about two hours at about 60° C. In one embodiment, hordein is precipitated and freeze-dried.

In one embodiment, in step (b), glutelin is treated with an alkaline solution. In one embodiment, in step (b), hordein is treated with an alcohol/alkaline solution. In one embodiment, in step (b), the reaction temperature is between about 40° C. to about 60° C., and the time of reaction is between about 10 minutes to about 120 minutes. In one embodiment, in step (c), the deamidated protein is recovered in the form of a freeze-dried protein concentrate.

In another aspect, the invention comprises deamidated glutelin produced by the above method and having a degree of deamidation ranging between about 0.5% to about 40%. In one embodiment, the degree of deamidation ranges between about 1.0% to about 15%. In one embodiment, the degree of deamidation ranges between about 1.0% to about 2.5%.

In another aspect, the invention comprises deamidated hordein produced by the above method and having a degree of deamidation ranging between about 0.7% to about 40%. In one embodiment, the degree of deamidation ranges between about 2.4% to about 4.7%.

In another aspect, the invention comprises a food, cosmetic or pharmaceutical product comprising the deamidated barley protein produced by the above method.

In yet another aspect, the invention comprises use of deamidated barley protein produced by the above method in a food, cosmetic or pharmaceutical product.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings:

FIG. 1 is a graph showing solubility of deamidated glutelin at different pHs as a function of DD.

FIG. 2 is a graph showing emulsion capacity and stability for deamidated glutelin.

FIG. 3 is a graph showing a time-dependent increase of DD of hordein induced by alkaline reaction.

FIG. 4 is a graph showing electrophoretic mobilities of deamidated hordeins at different pH as a function of DD.

FIG. 5 is a graph showing degree of hydrolysis and surface hydrophobicity of deamidated hordeins as a function of DD.

FIG. 6 is a photograph of a SDS-PAGE gel showing deamidated hordeins (a: unmodified hordein; b: DD 0.7%; c: DD 1.2%; d: DD 4.7%; e: DD 9.8%).

FIG. 7 shows SEC-HPLC chromatograms of deamidated hordeins (a: unmodified hordein; b: DD 0.7%; c: DD 1.2%; d: DD 4.7%; e: DD 9.8%; f: DD 17%).

FIG. 8 shows FTIR spectra of deamidated hordeins (a: DD 1.2%; b: DD 4.7%; c: DD 9.8%; d: DD 17%).

FIG. 9 is a graph showing solubility of deamidated hordeins at different pHs as a function of DD.

FIGS. 10A-C are graphs showing foaming capacity and stability of deamidated hordeins at pH 3 (FIG. 10A); pH 5 (FIG. 10B); and pH 7 (FIG. 10C) as a function of DD.

FIGS. 11A-C are graphs showing emulsion centrifugation stability and emulsion thermal stability of deamidated hordeins at pH 3 (FIG. 11A); pH 5 (FIG. 11B); and pH 7 (FIG. 11C) as a function of DD.

### DETAILED DESCRIPTION OF PREFERRED EMBODIMENTS

The present invention relates to partially deamidated barley proteins. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

The present invention relates to partially deamidated barley proteins having various degrees of deamidation (DD), and methods for preparing same. In one embodiment, the deamidated barley protein is glutelin. In one embodiment, the deamidated barley protein is hordein. As used herein, “hordein” refers to prolamin proteins which may be extracted from barley with ethanol, and includes hordeins A, B, C and D.

As used herein, the term “deamidation” refers to a chemical reaction in which an amide functional group is removed from an polypeptide. The primary targets of deamidation are asparagine and glutamine residues.

In one embodiment, the invention comprises a method of producing a deamidated protein from barley, comprising the steps of:

a) extracting protein from barley;

b) treating the extracted protein with an alkaline or acidic solution for a sufficient time and temperature for deamidation; and

c) recovering deamidated protein from the solution, wherein the deamidated protein has improved functional properties with respect to solubility, and emulsifying and foaming properties at acidic and neutral pHs compared to those of a non-deamidated protein.

The deamidated proteins are produced from barley using the methods described herein. The method generally involves at least the steps of extracting protein from barley using an alkaline solution or an alcohol; treating the extracted protein with an alkaline or acidic solution for a sufficient time and temperature for deamidation; and recovering deamidated protein from the solution. The physicochemical properties of the resultant deamidated barley proteins may have functional properties and suitability for particular applications such as, for example, as emulsion and foam stabilizers in food, cosmetic and pharmaceutical products.

The detailed steps of the process are as follows. Barley is used as the starting material. As used herein, the term “barley” means a grass in the genus Hordeum. Barley proteins may be extracted using the method described by Wang et al. (2010). In one embodiment, the barley protein is extracted using a suitable extractant. Hordein is soluble in alcohol, while glutelin is insoluble in alcohol. Glutelin may be extracted with an alkaline solution from the barley fraction which is insoluble in alcohol.

In one embodiment, barley is pearled and milled. Pearling is used to remove the grain's outer layers (mainly bran and germ) so that the barley cytoplasmic proteins (albumin and globulin) are enriched in the pearling flour, while endosperm proteins (hordein and glutelin) are enriched in the pearled grain flour. The pearled grain flour is treated with an alcohol. In one embodiment, the alcohol is ethanol, methanol or propane, and is preferably a 50%-70% ethanol. In one embodiment, the ratio of alcohol to flour is 6:1 (v/w). In one embodiment, the solution is stirred for about two hours at about 60° C. After extraction, the insoluble solids are separated by centrifugation. The alcoholic supernatant is collected, and hordein is isolated by cold precipitation and may be freeze-dried for storage and future use.

The barley endosperm flour residue which is insoluble in alcohol is mixed with an alkaline solution to extract glutelin. In one embodiment, the alkaline solution is a solution of NaOH and has a pH of about 10 or higher. In one embodiment, the ratio of the alkaline solution to flour is 10:1 (v/w). In one embodiment, the mixture is stirred for about half an hour at about room temperature. After extraction, the insoluble solids are separated by centrifugation. The supernatants collected from the alkaline extracts are adjusted to an acidic pH to precipitate the proteins. Glutelin protein isolates are then obtained by centrifugation and may be freeze-dried for storage and future use.

The barley proteins are modified to form deamidated proteins having desirable physical and chemical properties (Examples 4 and 5). Barley proteins may be deamidated by an alkaline or an acidic process. In one embodiment, the alkaline method is preferred because it is considered to be more efficient than an acidic method. However, barley proteins deamidated by an acidic method or by other methods are considered within the scope of this invention.

In one embodiment, glutelin is deamidated by treatment with an alkaline solution at an elevated temperature. In one embodiment, the alkaline solution comprises a 0.1M to about 1.0M sodium hydroxide solution. In one embodiment, the temperature is about 40° C.

In one embodiment, hordein is deamidated by treatment with an alcohol/alkaline solution at an elevated temperature. In one embodiment, the solution comprises 70% ethanol with 0.1M to about 1.0M sodium hydroxide. In one embodiment, the temperature is between about 40° C. to about 60° C.

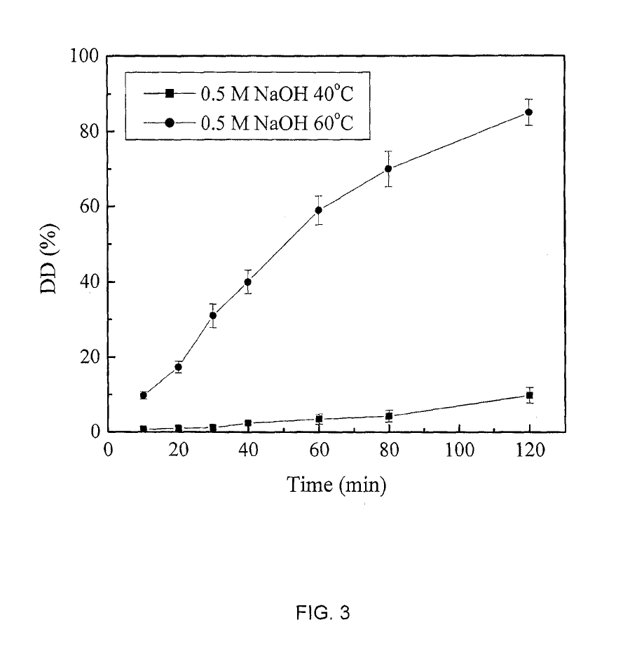

A greater DD may be achieved with a stronger alkaline solution, longer reaction time and/or higher temperatures. Using hordein as an example, FIG. 3 shows the DD obtained as a function of the reaction time. At 40° C., the DD reached 9.8% after two hours. Prolonged time did not further increase the DD. Increasing the temperature to 60° C. significantly enhanced the reaction rate and the DD reached more than 80% within two hours. Samples having DD values in the range of 0.7-9.8% and 17-40% which had been obtained at 40° C. and 60° C. respectively, were selected for further study (Table 1).

TABLE 1

| | NaOH | Time | Temperature | DD |
|---|---|---|---|---|
| Sample | (M) | (min) | (° C.) | (%) |
| 1 | 0.5 | 10 | 40 | 0.7 |
| 2 | 0.5 | 30 | 40 | 1.2 |
| 3 | 0.5 | 40 | 40 | 2.4 |
| 4 | 0.5 | 80 | 40 | 4.7 |
| 5 | 0.5 | 120 | 40 | 9.8 |
| 6 | 0.5 | 20 | 60 | 17 |
| 7 | 0.5 | 30 | 60 | 31 |
| 8 | 0.5 | 40 | 60 | 40 |

One skilled in the art may thus produce deamidated barley proteins with a desired DD by varying the strength of the alkaline solution, the reaction time and/or temperature. In one embodiment, the reaction temperature is between about 40° C. to about 60° C. In one embodiment, the time of reaction is between about 10 minutes to about 120 minutes.

After a desired time, the reaction is halted by addition of acid or alkaline, as the case may be. In one embodiment, the acid comprises hydrochloric acid. The solution is dialyzed against deionized water and the deamidated protein is recovered in the form of a freeze-dried protein concentrate.

The physicochemical properties of the resultant deamidated barley proteins may be evaluated to assess their suitability for particular applications. Such properties may vary as a function of the DD, and may include, but are not limited to, degree of hydrolysis (Example 6), solubility (Example 7), foaming properties (Example 8), emulsion properties (Example 9), electrophoretic mobility (Example 10), surface hydrophobicity (Example 11), molecular weight (Examples 12 and 13), and secondary structure (Example 14). Since proteins are amphoteric polyelectrolytes, emulsifying and foaming behaviours are expected to vary with pH; thus, the impact of pH on various functional properties, including emulsifying and foaming behaviors, may be assessed as described herein.

The following are specific examples of embodiments of the present invention. These examples demonstrate exemplary deamidated barley proteins, namely glutelin and hordein.

These examples are offered by way of illustration and are not intended to limit the invention in any manner.

In one embodiment, the invention comprises deamidated glutelins having various DD which result in enhanced solubility while maintaining the main glutelin structures, and improved emulsifying and foaming properties. In one embodiment, the invention comprises deamidated glutelins produced by the above method and having a DD ranging between about 0.5% to about 40%. In one embodiment, the DD ranges between about 1.0% to about 15%. In one embodiment, the DD ranges between about 1.0% to about 2.5%.

The DD, degree of hydrolysis (HD) and surface hydrophobicity for exemplary deamidated glutelins are set forth in Table 2:

TABLE 2

| | | | | Degree of | Degree of | Surface |
|---|---|---|---|---|---|---|
| | NaOH | Time | Temperature | deamidation | hydrolysis | hydrophobicity |
| Sample # | (M) | (mins) | (° C.) | (%) | (%) | (cm²/ml) |
| 1 | 0.1 | 30 | 40 | 0.05 | 14.51877 | 132.6 |
| 2 | 0.1 | 40 | 40 | 0.07 | 16.1535 | 150 |
| 3 | 0.1 | 50 | 40 | 1.18 | 16.39774 | 169.98 |
| 4 | 0.1 | 80 | 40 | 2.23 | 19.24648 | 202.88 |
| 5 | 0.5 | 40 | 40 | 15 | 21.01847 | 189.935 |
| 6 | 0.5 | 90 | 40 | 30.0 | 22.6259 | 147.27 |
| 7 | 0.5 | 120 | 40 | 40.0 | 17.28763 | 149.14 |

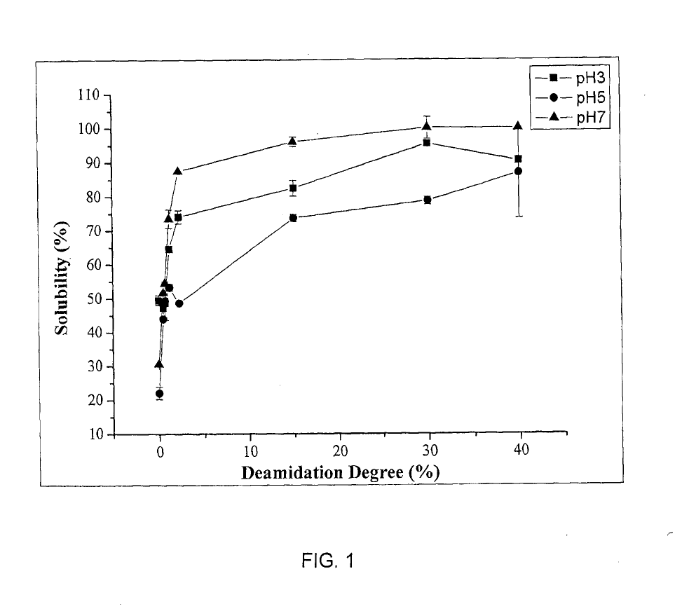

Unmodified glutelin shows low solubility (less than 20%) at pH 3-7 and is soluble in water only in the presence of high concentrations of alkali (pH 11) due to the high proportion of nonpolar amino acid residues and high surface hydrophobicity (data not shown). However, the solubility of deamidated glutelin increases significantly at acidic and neutral pHs (FIG. 1). The remarkably improved solubility after deamidation at both acidic and neutral pHs will enable a broader range of glutelin usage in various applications.

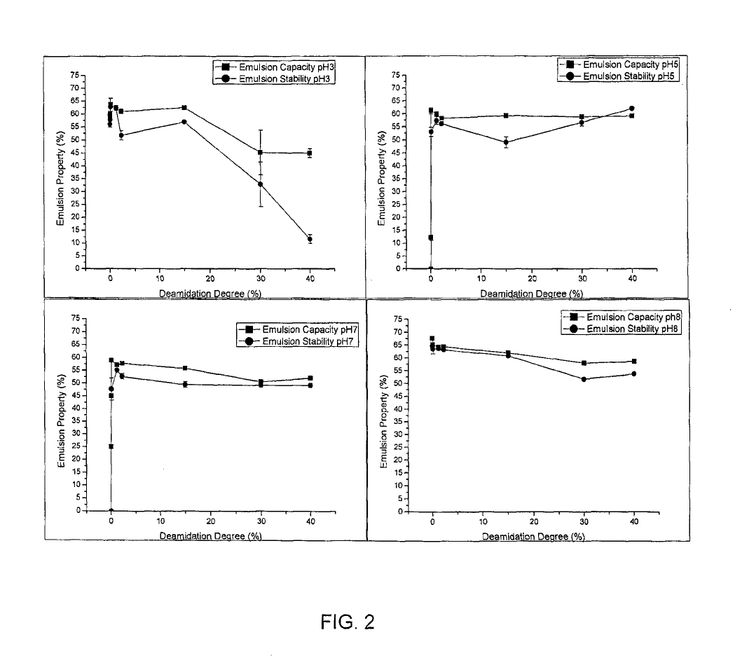

Unmodified glutelin shows good emulsifying stability at both acidic and neutral conditions (data not shown). These data were obtained by dehydrating unmodified glutelin at pH 11, followed by adjusting the pH back to acidic and neutral conditions before evaluating the emulsifying property. For the present invention, the emulsifying stability was evaluated by dispersing deamidated glutelins directly in buffer at different pHs. FIG. 2 shows the emulsion centrifugation capacity and emulsion thermal stability of deamidated glutelin having different DD. Deamidated glutelin demonstrates an excellent capacity to stabilize the emulsion at a broad range of DD since around 60-65% of formed emulsions remained even after heating and centrifugation. Without restriction to any theory, this favorable property is likely due to glutelin's unique molecular structure, strong surface hydrophobic, and tendency to form aggregates.

In one embodiment, the invention comprises deamidated hordeins having various DD which result in dissociated hordein aggregates. In one embodiment, the invention comprises deamidated hordein produced by the above method and having a DD ranging between about 0.7% to about 40%. In one embodiment, the DD ranges between about 2.4% to about 4.7%. Optimum functionalities are obtained in a DD range between about 2.4% to about 4.7%, where hordein demonstrates improved solubility, and emulsifying and foaming properties at both acidic and neutral pHs. A DD greater than about 4.7% results in extensive protein hydrolysis and a marked change in protein secondary structure which greatly influences functionality.

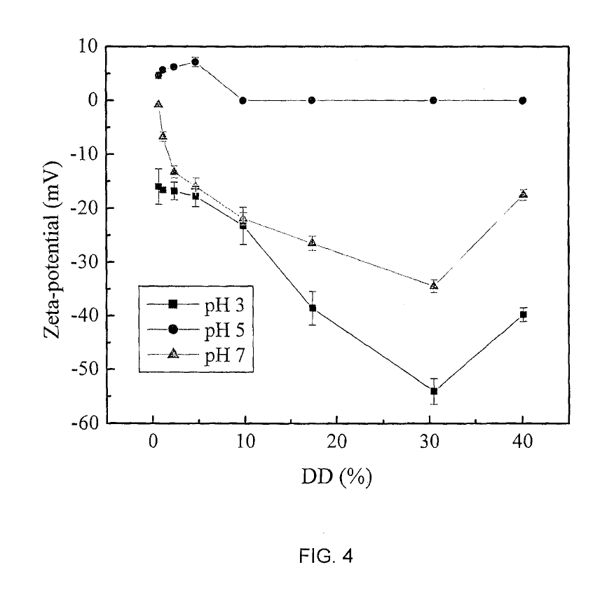

The electrophoretic mobilities of deamidated hordeins at different pHs were determined (FIG. 4). The zeta-potential of deamidated hordeins in different pH buffers are expressed as a function of DD. Limited surface charge (−5 mV) is observed for hordein with a DD of 0.7% at pH 5. This value, however, increases to −33 mV at a DD of 31%, and then decreases to −17 mV at a DD of 40%. The surface charge of the deamidated hordein changes in the same manner at pH 7, but the zeta-potential is generally higher than that at pH 5, especially at relatively low or high DD range. Conversely, the protein molecule surfaces are slightly positively charged (+5 mV) at pH 3 when the DD is 0.7%. With increasing of the DD to 4.7%, hordein surface charge decreases to near zero. Without restriction to a theory, hordein may have an aggregation structure similar to gliadins. The isoelectric point (IEP) of hordein (without deamidation) is between pH 3 and 5. As the DD increases, the IEP shifts from about pH 5 to about pH 3 due to the introduction of additional carboxyl groups on the protein side chains as a result of deamidation. The IEPs of other proteins similarly shift to acidic pH after deamidation (Matsudomi et al., 1985). These data also explain the increase of the protein surface charge with DD until 31% at pH 5 and 7. The decrease of zeta-potential at a DD of 40% in both pH 5 and 7 buffers is unexpected, and may be attributed to cleavage of the small peptides with high density of charge due to significant hydrolysis. These small peptides are lost during dialysis, even though very low molecular weight cut-off (1 kDa) dialysis tubing is used.

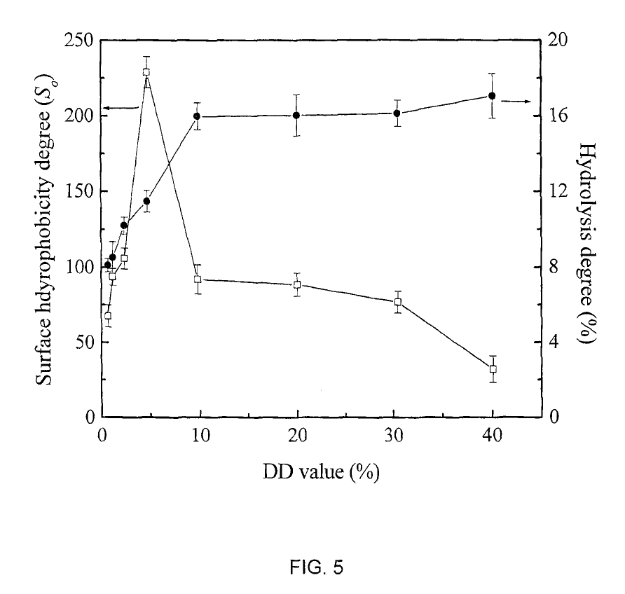

FIG. 5 shows the HD and surface hydrophobicity of deamidated hordeins. The HD increases linearly in proportion to the DD until a DD of 9.8%, after which the HD value levels off This result suggests that hordein peptide bond cleavage occurs rapidly within a DD ranging between about 0.7% to about 9.8%, and the hydrolysis rate slows after a DD of 9.8%. The degree of surface hydrophobicity of the deamidated hordein increases markedly as the DD increases to about 4.7%, suggesting that the hydrophobic regions are progressively exposed at the molecular surface. A further increase in the DD results in a significant decrease in surface hydrophobicity. A DD greater than about 5% thus leads to protein unfolding and extensive hydrolysis. Since more polar groups on protein side chains are exposed, a decrease in surface hydrophobicity is observed.

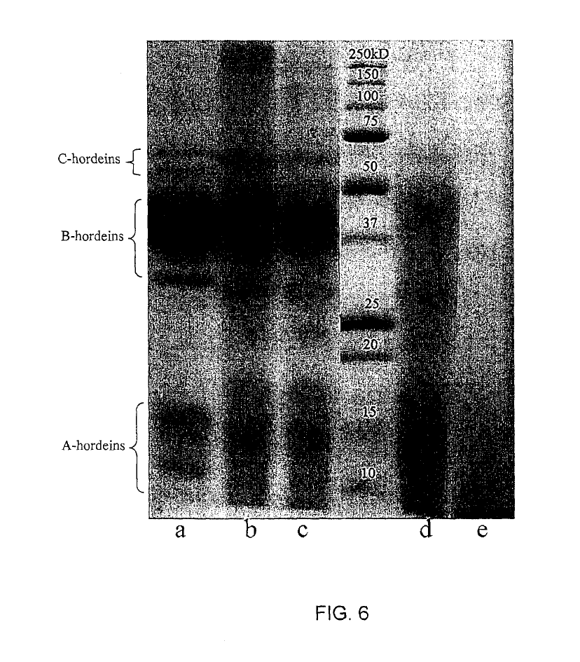

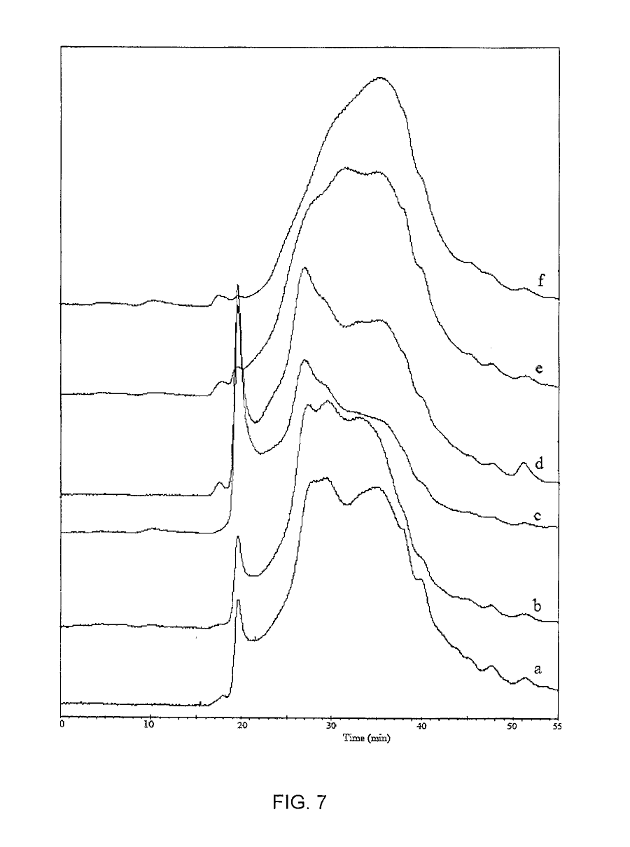

FIG. 6 shows SDS-PAGE of deamidated hordeins. Three subunits of hordein were identified with bands at 55-80, 30-50 and less than 15 kDa corresponding to C, B and A hordeins, respectively. A weak band at 80-90 kDa corresponding to D hordein is observed when ethanol is used as the sole extraction agent (Bilgi et al., 2004). Most bands remain visible in SDS-PAGE until a DD of 4.7%, but the band intensity of C and B hordeins decreases gradually. After a DD of 4.7%, all bands disappear. The result indicates that partial hydrolysis occurs at a DD less than or equal to 4.7%, whereas extensive hydrolysis occurs within a DD of between about 5% to about 9.8%, resulting in formation of peptides having molecular weights of less than 10 kDa.

The dissociation of large protein aggregates may lead to the formation of water soluble peptide aggregates. SEC chromatograms of deamidated hordeins in phosphate buffer are shown in FIG. 7. Deamidated hordein with a DD of 0.7% contains two main broad peaks (peak 1 and peak 2) corresponding to subunits having molecular weights of less than 15 kDa and 20-67 kDa, respectively. The former can be assigned to A hordeins, whereas the latter could be B and C hordeins together. A small sharp peak (peak 3) was also observed at 114 kDa, which could be assigned to some aggregated large peptides. This phenomenon has been reported for barley proteins, where high molecular weight subunits form a backbone which binds low molecular weight subunits through disulfide bridges to form a gel-like aggregate (Celus et al., 2006). Increasing the DD to between about 2.4 to about 4.7% significantly alters the chromatogram patterns. Peak 2 is markedly sharpened and peak 3 amplitude is dramatically enhanced. The sharpened peak 2 corresponds to the remaining of more hydrolysis-resistant subunits, likely corresponding to C-hordeins since they are more slowly degraded than B-hordeins (Celus et al., 2006). The increased peak 3 intensity can be attributed to an increased solubility of the large polypeptides due to an increased net negative charge by deamidation (Chan et al., 1999). A further increase of DD value equal or greater than 9.8% results in the dissociation of the aggregated large peptides as the peak 3 almost disappears. An obvious shift of peak 2 to lower molecular weight range is observed, indicating that the resistant subunits in hordein start to be hydrolyzed after a DD of 4.7%. The degradation may account for the extensive hydrolysis of deamidated hordeins within a DD range of between about 5% to about 9.8%.

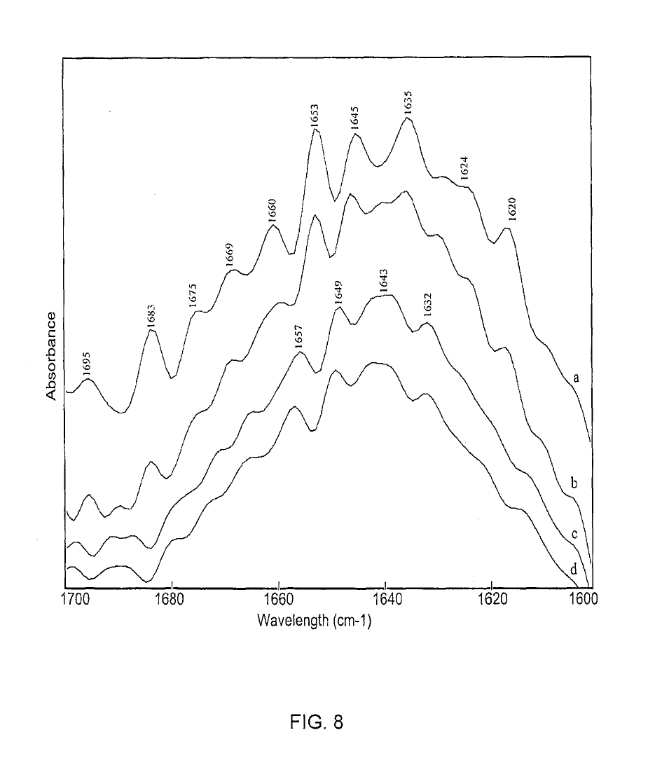

Secondary structure may be determined by Fourier transform infrared spectroscopy (FTIR). Through proper fitting of the amide I band of the original FTIR spectrum of a protein, the conformation of the protein (i.e., helix, sheet or turn) can be obtained. Hordein having a DD of 0.7% shows several bands in the amide I region (FIG. 8). Such bands represent protein secondary structures in accordance with previous reports (Liu et al., 2009; Mejia et al., 2007; Siu et al., 2002; Wellner et al., 1996; Yong et al., 2006): α-helices (1652 cm⁻¹), β-sheets (1617, 1635 and 1683 cm⁻¹), (3-turn (1669 and 1675 cm⁻¹), and random coils (1646 cm⁻¹). The band at 1660 cm⁻¹ could be mainly assigned to the carbonyl stretching of the glutamine side chain. The bands at 1683 and 1917 cm⁻¹ are believed to be associated with the aggregation process. When the DD increases from about 2.4% to about 4.7%, the intensity of the bands at both 1683 and 1917-1621 cm⁻¹ decreases, suggesting disassociation of protein aggregates, probably due to increased repulsions between protein molecular chains as a resulted of increase surface charges. Consequently, more hydrophobic patches on protein unit surfaces are exposed outside, thus increasing the surface hydrophobicity. Marked shifts in the band positions in the wavelength range of 1623-1657 cm⁻¹ are observed with a further increase of the DD. The absorption corresponding to the glutamine side chain shifts to 1656 cm⁻¹, reflecting a change of intra- or inter-molecular hydrogen bonds between glutamine side chains (Wellner et al., 1996). Additionally, the a-helix band shifts to lower wavelength and the random coil band (about 1642 cm⁻¹) intensity increases notably. This suggests that marked protein confirmation changes occur following a DD value of 4.7%, likely associated with protein partial unfolding as a result of both strong negative charge on protein molecular chains and extensive protein hydrolysis.

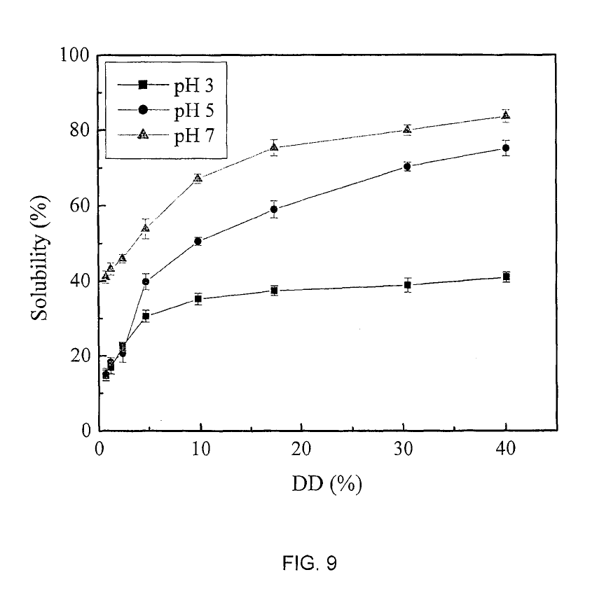

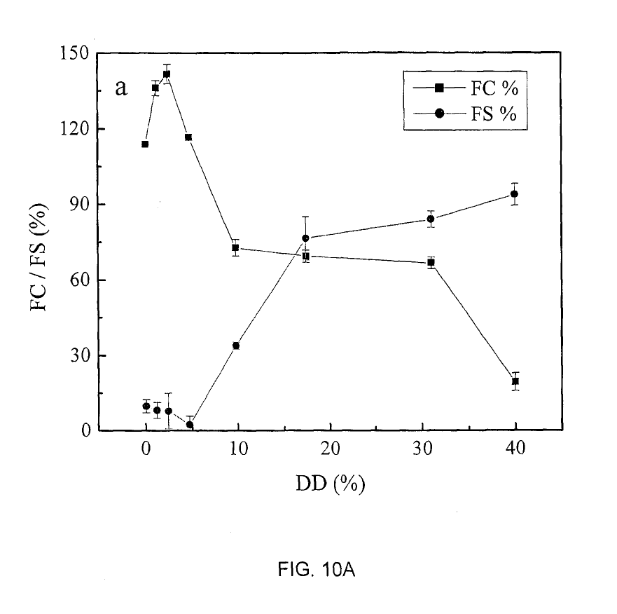

Unmodified hordein shows low solubility (less than 20%) at pH 3-7 and a significant increase in solubility (about 50%) at pH 10 (Wang et al., 2010). Due to the high proportion of nonpolar amino acid residues and high surface hydrophobicity, unmodified hordein is soluble in water only in the presence of alcohol, high concentrations of urea, high concentrations of alkali (pH 11), or anionic detergents, similar to other prolamin proteins (Shukla and Cheryan, 2001). The solubility is relatively low at pH 3 due to shift of the hordein isoelectric point to acidic pH. In comparison to unmodified hordein, deamidated hordeins exhibit improved solubility at both acidic and neutral pHs (FIG. 9). Protein solubility at pH 5 increases from 15% to 75% as the DD increases to 40%. Without restriction to a theory, the improvement in solubility within a DD of between about 0.7% to about 4.7% may be attributed to the dissociation of hordein aggregates and partial protein hydrolysis. Further increased solubility after a DD of 4.7% may be due to protein partial unfolding and extensive hydrolysis. Such structural changes lead to the exposure of more charged and polar groups to the surrounding water, thus promoting protein-water interaction and an increased solubility (Chan and Ma, 1999).

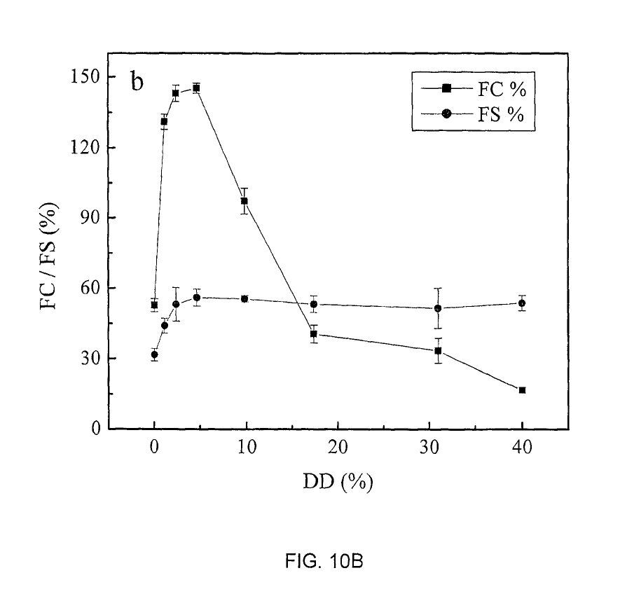

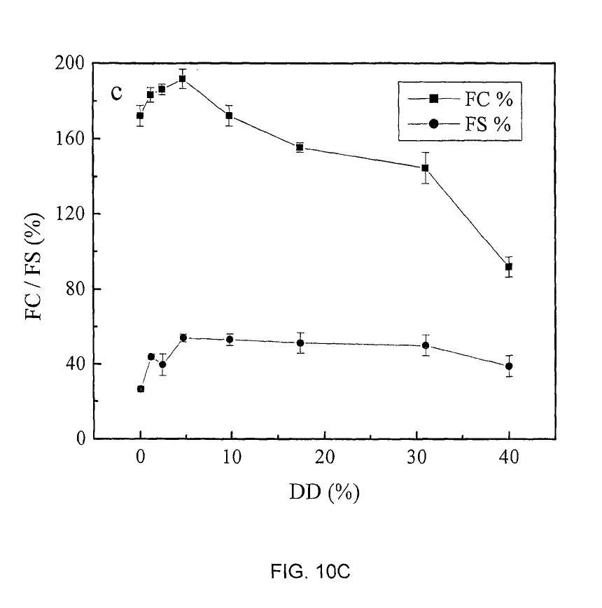

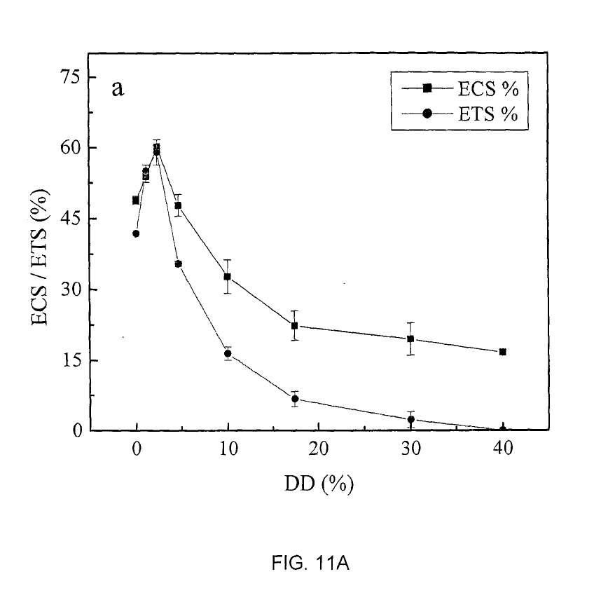

Unmodified hordein shows good foaming capacity at both pH 3 and neutral conditions (150-160%), but a relatively lower foaming capacity at pH 5 (90%). However, due to its inherent poor solubility, unmodified hordein requires dehydration at pH 11 followed by adjusting pH back to acidic and neutral conditions to enable foaming and emulsifying functionalities. This procedure is impractical in commercial food systems. However, deamidation significantly improves hordein solubility even within a limited DD range, thus allowing functionality testing by dispersing samples at different pH buffers directly. Hordein having a DD between about 2.4% to about 4.7% has improved foaming capacity (FC) at pH 5 (145%) and pH 7 (190-200%) compared to unmodified hordein (FIGS. 10A-C). With increasing DD, the FC initially increases until a DD between about 2.4% to about 4.7%, then decreases at acidic and neutral pHs. A much more rapid decrease in FC is observed at pH 3 and pH 5 than pH 7. The optical FC values were obtained at a narrow DD range (about 2.4% to about 4.7%) where a significant improvement of FC was observed at pH 5 (145%) and pH 7 (190-200%) compared to unmodified hordein. The optical FC obtained at pH 3 is on a same level as that of the unmodified sample.

Without restriction to a theory, the initial increase of the FC value within a DD between about 0.7% to about 4.7% may be due to an increase of the protein solubility, enabling diffusion to the air/water surface. The exposed hydrophobic side chains facilitate binding of deamidated hordein at hydrophobic air surfaces, and these proteins may aggregate via surface hydrophobic patches to form films around bubble surfaces. Although deamidated hordeins exhibit good solubility when the DD is greater than about 4.7%, their surface hydrophobicity decreases with a further increase of the DD. This decreased protein surface hydrophobicity may be one of the major reasons accounting for the decreased FC values at the DD range of about 9.8% to about 40%.

The protein surface charge also influences the FC value. A significantly greater FC is observed at pH 7 compared to pH 3 and 5 within the optical DD range. Without restriction to a theory, this may be related to a greater surface charge on protein molecular chains at neutral pH, resulting in a strong repulsion between adjacent bubbles, and preventing quick foam coalescence during homogenization process. This greater surface charge could also explain the slower decrease in the rate of the FC values at pH 7 after a DD of about 4.7%.

Deamidated hordein shows an increased foaming stability (20% to 50-60%) at both pH 5 and 7 when the DD value is raised from about 0.7 to about 4.7%, and then levels off after a DD of about 4.6% (FIGS. 10B-C). Low FS values are observed at a DD range of about 0.7 to about 4.7% at pH 3, and this value increased rapidly after a DD of 4.7% (FIG. 10A).

Without restriction to a theory, the overall stability of foam is related to the resistance of the lamella to drain and of the bubbles to collapse. These factors are dependent on the rheological and adhesive properties of the interfacial film surrounding the bubble (German et al., 1985). Normally high molecular weight proteins exhibit greater film strength and foam stability; thus, the aggregated large peptides observed in the SEC chromatograms at the DD range of about 2.4 to about 4.7% may have contributed to the increased hordein foaming stability at both pH 5 and 7. However, the FS values did not decrease after DD value of about 4.7%. This is difficult to explain because large peptide aggregates were dissociated at a DD equal or greater than 9.8% according to the SEC chromatograms. The FC values decreased significantly after a DD of 4.7% at both pH 5 and 7. It is deduced that fewer protein molecular chains have suitable molecular structures for foam forming compared to hordeins with DD of about 2.4% to about 4.7%. However, once foams were prepared, the protein chains with a suitable molecular structure could form continuous, rather rigid films around bubbles, which might explain why the ES value remained almost unchanged at a DD value equal or greater than about 9.8%. Optical foaming stability is observed near the protein IEP. Proteins can adsorb better to the air/water interface at minimum electrostatic repulsion to form a rigid film against coalescence (German et al., 1985). However, a very low FS value (equal to or less than 9.8%) was observed for deamidated hordeins at a DD range of 0.7-4.7% when the pH was 3. It is assumed that the deamidated hordeins, including the large peptide aggregates, also formed a thick and rigid film at air/water interface at pH 3. This film may have a strong tendency to aggregate when the surface charge is low, resulting in extensive aggregation of protein films between adjacent gas bubbles, film rupture, and foam instability. After increasing the DD to equal or greater than 9.8%, large peptides were dissociated and the hordein peptide bonds were cleaved. The hydrolyzed peptides may exhibit fewer tendencies to aggregate, forming rigid and viscoelastic films without aggregation near protein IEP. This explains a significant increase of the FS value observed with a DD value increasing from 9.8 to 40% at pH 3.

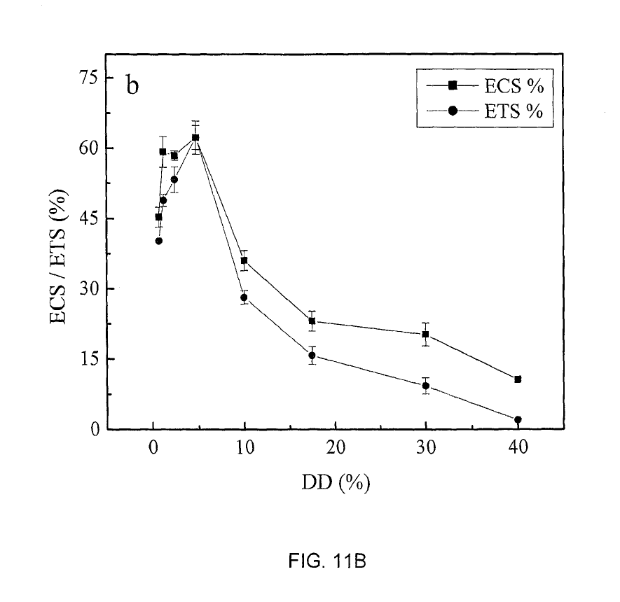

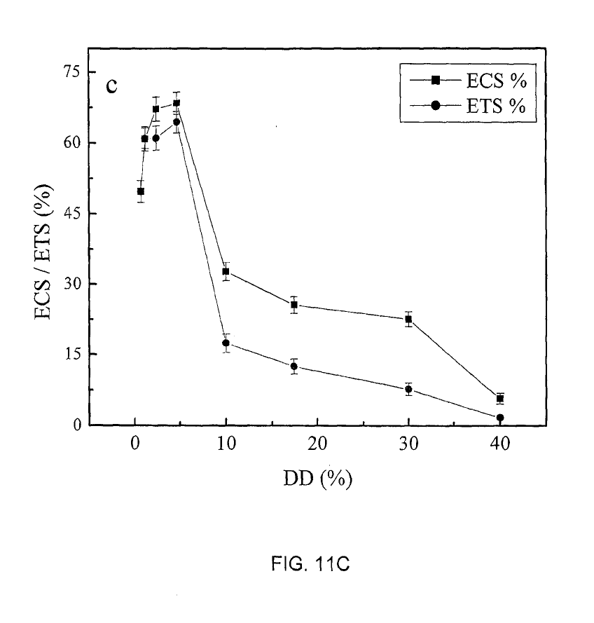

Unmodified hordein shows good emulsifying stability at pH 3 and neutral conditions (ECS 57-61%, ETS 51%), where low emulsifying property was observed at pH 5 (ECS 31%, ETS 18%) (Wang et al., 2010). These data were obtained by dehydrating unmodified hordein at pH 11 followed by adjusting pH back to acidic and neutral conditions before evaluating their foaming property. Emulsifying stability may be evaluated by dispersing deamidated hordeins directly in buffer at different pHs. An increase of the ECS is observed until the DD of between about 2.4 to about 4.7% at all pHs (FIGS. 11A-C). After a DD of about 4.7%, the ECS value decreases rapidly. The change of the ETS as a function of the deamidation degree follows the same trend.

The initial increase of the ECS can be attributed to the increase in solubility and exposed hydrophobic side chains, since protein solubility and hydrophobicity have a strong correlation with emulsifying properties (Nakai, 1983; Townsend and Nakai, 2006). The aggregated large peptides observed in the SEC chromatograms may also play a role in stabilizing the emulsions. Large peptides can generally form a rigid film at the oil/water interface to prevent the close contact of oil droplets, and decrease flocculation and coalescence (Agyare et al., 2009; McClements, 1999). A further increase of the DD equal or greater than about 9.8% resulted in decreased surface hydrophobicity due to protein unfolding to expose the polar side chains, dissociation of the large peptides, and extensive protein hydrolysis. Such factors would prevent the formation of a continuous protein film at the oil-liquid interface, leading to reduced emulsion stability. Deamidated hordein demonstrates an excellent capacity to stabilize the emulsion at a DD of between about 2.4% to about 4.7% since approximately 70% of the formed emulsions remained after heating and centrifugation. This favorable property is likely due to hordein's unique molecular structure, strong surface hydrophobicity, and tendency to form aggregates. The excellent emulsion thermal stability may be due to further gelation of the deamidated hordein around the oil droplets during thermal treatment to form a reinforced film.

Accordingly, the deamidated barley proteins produced by the methods described herein may be used for example, as emulsion and foam stabilizers in food, cosmetic and pharmaceutical products. In one embodiment, the invention comprises use of deamidated barley proteins in a food, beverage, cosmetic or pharmaceutical product. In one embodiment, the invention comprises a food, beverage, cosmetic or pharmaceutical product comprising the deamidated barley proteins. As examples, the emulsifying property of deamidated glutelin and the foaming property of deamidated hordein may support their development as functional ingredients in food formulations such as whipped topping, salad dressing, and processed meats.

In other embodiments, the invention may comprise products such as nutraceuticals, agricultural, personal care, paint, ink, coatings, detergent, soap, or firefighting compositions, which comprises a deamidated barley protein.

Exemplary embodiments of the present invention are described in the following Examples, which are set forth to aid in the understanding of the invention, and should not be construed to limit in any way the scope of the invention as defined in the claims which follow thereafter.

### EXAMPLE 1

### Materials

Regular barley grains (Falcon) were provided by James Helm, Alberta Agricultural and Rural Development, Lacombe, Alberta, Canada. Protein content was 13.2% (w/w) as determined by combustion with a nitrogen analyzer (FP-428, Leco Corporation, St. Joseph, Mich., USA) calibrated with analytical reagent-grade EDTA and a protein calculation factor of 6.25. Canola oil used for the emulsifying study was purchased locally. Unstained standard protein molecule marker for SDS-PAGE was purchased from Bio-RAD (Richmond, Calif., USA). An Ammonia Assay Kit, o-phthaldialdehyde reagent, 1-anilinonaphthalene-8-sulfonic acid and standard molecular markers for HPLC analysis (BSA, 67 kDa; ovalbumin, 43 kDa; lactoglobulin, 35 kDa; cytochrome C, 13.6 kDa; aprotinin, 6.5 kDa and vitamin B₁₂, 1.4 kDa) were purchased from Sigma-Aldrich Canada Ltd. (Oakville, ON, Canada). All other chemicals were of reagent grade.

### EXAMPLE 2

### Extraction of Hordein

Barley hordein was extracted according to Wang et al. (2010). After pearling and milling, barley endosperm flour was dispersed in the 55% ethanol solution at a solvent-to-flour ratio of 6:1 (v/w) with stirring for two hours at 60° C. After extraction, the solids were separated by centrifuge (8,500×g for 15 min at 23° C.) (Beckman Coulter Avanti™ J-E Centrifuge System, USA). The hordein fraction was isolated from the supernatant by cold precipitation at 4° C. overnight. The isolated hordein was freeze-dried and refrigerated at 4° C.

### EXAMPLE 3

### Extraction of Glutelin

Barley glutelin was extracted according to Wang et al. (2010). After alcohol extraction of the hordein fraction, the remaining barley endosperm flour was dispersed in the pH 11 alkaline solution adjusted using 0.1 M NaOH solution at a solvent-to-flour ratio of 10:1 (v/w) with stirring for 0.5 h at room temperature. After extraction, the insoluble solids were separated by centrifuge (8,500 x g for 15 min at 23° C.) (Beckman Coulter Avanti™ M J-E Centrifuge System, USA). The supernatants were adjusted to approximately pH 5 with 0.5 M HCl to precipitate the proteins. Protein isolates were obtained by centrifugation at 8,500×g for fifteen minutes at room temperature. The protein fractions were freeze-dried and refrigerated at 4° C. Protein content of the protein fractions was determined using a nitrogen analyzer (FP-428, Leco Corporation, St. Joseph, Mich., USA).

### EXAMPLE 4

### Preparation of Deamidated Barley Proteins

Deamidated barley proteins of different DD were prepared according to Yong et al. (2004) with modifications. Glutelin (5%, w/v) was suspended in 0.1-0.5 M NaOH solution at 40° C. under constant stirring. Hordein (5% w/v) was suspended in 70% (v/v) ethanol with 0.5 M NaOH solution at 40 and 60° C. All samples were withdrawn at different time intervals (10-120 minutes) to provide a relatively broad range of DD values (particularly those within limited DD range of less than or equal to 10%), and neutralized using 0.5 M HCl before dialysis against deionized water and then freeze dried. The dried samples were stored at 4° C. until use.

### EXAMPLE 5

### Determination of the Degree of Deamidation (DD)

The DD was determined by measurement of the released ammonia after deamidation using an Ammonia Assay Kit according to the manufacturer's instructions. DD was calculated as the ratio of ammonia generated in the modified sample to that of the completely deamidated protein. Complete deamidation was achieved by refluxing the sample with 2 M HCl for two hours.

### EXAMPLE 6

### Determination of the Degree of Hydrolysis (HD)

The HD was assayed by quantifying cleaved peptide bonds using o-phthaldialdehyde (OPA) (Paraman et al., 2007). The OPA reagent was prepared by dissolving 7.62 g of disodium tetraborate decahydrate and 200 mg of SDS in 150 mL of deionized water, followed by addition of 160 mg of OPA dissolved in 4 mL of ethanol and 176 mg of 99% dithiothreitol. The volume of the mixture was adjusted to 200 mL using deionized water. OPA reagent (3 ml) was mixed with 400 μL of deamidated hordein sample (10 mg/mL) and the absorbance at 340 nm was measured using a spectrophotometer (Jenway 6505 UV/Vis Spectrophotometers, UK). The standard solution was prepared by dissolving 10 mg of serine into 100 mL of deionized water.

Deamidated glutelin (1.25 mg/mL) was dissolved in 12.5 mM of borate buffer (pH 8.5) plus 2% (w/v) of SDS. This solution (50 μL) was mixed with 1 mL of reagent (50 mL of 0.1 M borate buffer (pH 9.3), 1.25 mL of 20% (w/v) of SDS, 100 mg of N,N-dimethyl-2-mercaptoethylammonium chloride, and 40 mg of OPA dissolved in 1 mL of methanol). The mixture was left to stand for two minutes before measurement of the absorbance at 340 nm. The number of amino groups was determined with reference to the L-leucine standard curve (between 0.5 and 5 mM). The HD was calculated as:

HD (%)=[(α−ni)/nT]×100 (1)

where nT=the total number of amino groups in original glutelin after total hydrolysis with 6 M HCl for twenty-four hours; ni=the number of amino groups in glutelin; and α=the number of free amino groups measured in the deamidated glutelin.

### EXAMPLE 7

### pH Solubility Profile

Deamidated barley proteins (125 mg) were dispersed in 25 mL of buffer at pH 3, 5 and 7. The dispersions were mixed for 1 h at room temperature by magnetic stirring before centrifuging at 1,200×g (for hordeins) or 3,000 x g (for glutelins) for 20 min at 4° C. The supernatants were filtered through a Whatman No. 1 filter paper to obtain clear filtrates. The protein concentration in the filtrates was determined by dye assay (Bradford, 1976) with bovine serum albumin as the standard. The solubility was expressed as a percentage of the total protein content of the starting sample.

### EXAMPLE 8

### Foaming Properties

Foaming capacity (FC %) and foaming stability (FS %) was determined according to Ahmedna et al. (1999) with modifications. Protein samples (0.5%, w/v) were dispersed in 50 mL of buffer at pH 3, 5 and 7. The solution was mixed for two minutes with a homogenizer (PowerGen™ 1000, Fisher Scientific, Fairlawn, N.J., USA) at speed three. Volumes were recorded before and after homogenization using a graduated cylinder. The percentage volume increase was calculated as:

FC (%)=(Vf₂−Vf₁)/Vf₁×100 (2)

where Vf₁ and Vf₂ represent the volume of the protein solution and the formed foams before and after homogenization. FS (%) was determined as the volume of foam that remained after 0.5 hours at 23° C. expressed as a percentage of the initial foam volume:

FS (%)=Vf₂/Vf₁×100 (3)

### EXAMPLE 9

### Emulsion Properties

The emulsion centrifugation stability (ECS) and emulsion thermal stability (ETS) were determined according to Yasumatsu et al. (1972) with modifications. Barley protein samples (0.5%, w/v) were dispersed in 50 mL of buffer at pH 3, 5 and 7, followed by addition of 50 mL of canola oil. The mixture was homogenized at speed six (for glutelins) or speed three (for hordeins) for two minutes to form an emulsion (PowerGen™ 1000, Fisher Scientific, Fairlawn, N.J., US). The emulsion was then centrifuged at 1,500×g for five minutes. ECS (%) is calculated by measuring the volume of the emulsion remaining after centrifugation (Ve₁) and before (i.e., total volume or Veₜ) using a graduated cylinder and recorded as:

ECS (%)=Ve₁/Veₜ×100 (4)

The emulsion samples were then heated to 80° C. in a water bath for thirty minutes and cooled to 23° C. Upon cooling, these tubes were centrifuged at 1500×g for five minutes. The volume of the remaining emulsified fraction (Ve₂) was recorded. ETS (%) is calculated as:

ETS (%)=(Ve₁/Veₜ×100 (5)

### Example 10

### Electrophoretic Mobilities

The electrophoretic mobilities of the deamidated hordein samples in different pH buffers (pH 3 and 5: 0.2 M acetate buffer, pH 7: 0.2 M phosphate buffer) were measured by laser Doppler velocimetry using a Zetasizer™ NanoS (model ZEN1600, Malvern Instruments Ltd, UK). Electrophoretic mobility (i.e., velocity of a particle within an electric field) was related to the zeta potential (ζ) using the Henry equation (Liu et al., 2009):

$${U}_{E} = \frac{2 ɛ \times ζ \times f ( κ α )}{3 η} \qquad ( 6 )$$

where η=the dispersion viscosity; ε=the permittivity; and f (κα)=a function related to the ratio of particle radius (α) and the Debye length (κ). The results are reported as the average of at least three measurements. Typical standard deviations were less than ±3 mV. The same buffers were used in following studies.

### EXAMPLE 11

### Surface Hydrophobicity

Surface hydrophobicity of the deamidated hordeins in sodium phosphate buffer (pH 7) was determined using a fluorescence probe, 1-anilinonaphthalene-8-sulfonic acid, according to Kato and Nakai (1980). Fluorescence intensity (FI) was measured at wavelengths of 390 nm (excitation) and 470 nm (emission) using a fluorospectrometer (FP-6300, Jasco, Tokyo, Japan). The surface hydrophobicity degree (S₀) was calculated by linear regression analysis using the slope of the straight line obtained by plotting FI as a function of protein concentration.

### EXAMPLE 12

### Electrophoresis

SDS-PAGE was performed to evaluate hordein subunits in the barley protein fractions using a vertical mini-gel system (Mini-Protein™ Tetra Cell, Bio-Rad, Hercules, Calif., USA). Deamidated hordein samples were mixed with the loading buffer (0.125 M Tris-HCl, pH 6.8, 4% SDS (w/v), 20% glycerol (v/v), 0.05% 2-mercaptoethanol (v/v) and 1% bromophenol blue (w/v)) and then heated at 100° C. for 5 min. After cooling, 18 μL of sample (5 mg/mL) was loaded on 5% stacking gel and 12% separating gel and subjected to electrophoresis at a constant voltage of 80 V. After electrophoresis, the gels were stained with 0.1% (w/v) Coomassie Brilliant Blue-R-250 in water-methanol-acetic acid (4:5:1, v/v) for 30 mM and destained with water-methanol-acetic acid (4:5:1, v/v).

### EXAMPLE 13

### Size Exclusion Chromatography (SEC)

SEC chromatography was performed using a HPLC system (Varian ProStar, USA) combined with a size exclusion column (Superdex™ 200 10/300 GL, Amersham Biosciences, USA). 50 mM phosphate buffer containing 150 mM sodium chloride was used as a mobile phase at a flow rate of 0.4 mL/min at 25 ±0.5° C. 50 μL of sample solution was injected into HPLC system and the protein was monitored at a UV wavelength of 280 nm. Standard molecular markers were used to calculate the weight-average molecular weight of the deamidated hordeins.

### EXAMPLE 14

### FTIR Spectroscopy

Protein conformation was studied with a Fourier transform infrared (FTIR) spectroscopy (Varian FTS-7000, US) in the wavenumber range from 400 to 4000 cm⁻¹ during 128 scans, with 4 cm⁻¹ resolution. 5% deamidated hordein samples were dissolved in D₂O solution. To ensure complete H/D exchange, samples were prepared two days before and kept at 4° C. prior to infrared measurements. Samples were placed between two CaF₂ windows separated by 25 μm polyethylene terephthalate film spacer. To study the amide I region of the protein, Fourier self-deconvolutions were performed using the software provided with the spectrometer. Band narrowing was achieved with a full width at half maximum of 20 cm⁻¹ and with a resolution enhancement factor of 2.0 cm⁻¹.

### EXAMPLE 15

### Statistical Analysis

All experiments were performed at least in triplicate. Error bars on graphs represent standard deviations. Statistical significances of the differences were determined by Student's t-test. The level of significance was p<0.05.

As will be apparent to those skilled in the art, various modifications, adaptations and variations of the foregoing specific disclosure can be made without departing from the scope of the invention claimed herein.

### REFERENCES

The following references are incorporated by reference, where permitted, as if reproduced herein in their entirety. These references are also indicative of the level of skill in the art.
Agyare, K. K.; Addo, K. and Xiong, Y. L. (2009) Emulsifying and foaming properties of transglutaminase-treated wheat gluten hydrolysate as influenced by pH, temperature and salt. Food Hydrocoll. 23:72-81.
Ahmedna, M.; Prinyawiwatkul, W. and Rao, R. M. (1999) Solubilized wheat protein isolate: Functional properties and potential food applications. J. Agric. Food Chem. 47:1340-1345.
Bilgi, B. and [symbol] elik, S. (2004) Solubility and emulsifying properties of barley protein concentrate. Eur. Food Res. Technol. 218:437-441.
Bradford, M. M. (1976) Rapid and sensitive method for quantization of microgram quantities of protein utilizing principle of protein-dye binding. Analyt. Biochem. 72:248-254.
Cabra, V.; Arreguin, R.; Vazquez-Duhalt, R. and Farres, A. (2007) Effect of alkaline deamidation on the structure, surface hydrophobicity, and emulsifying properties of the Z19 r-zein. J. Agric. Food Chem. 55:439-445.
Celus, I.; Brijs, K. and Delcour, J.A. (2006) The effects of malting and mashing on barley protein extractability. J. Cereal Sci. 44:203-211.
Chan, W. M. and Ma, C. Y. (1999) Acid modification of proteins from soymilk residue (okara). Food Res. Int. 32:119-127.
Chiue, H.; Kusano, T. and Iwami, K. (1997) Antioxidative activity of barley hordein and its loss by deamidation. J. Nutr. Sci. Vitaminol. 43:145-154.
Food and Drug Administration (2005) Food labeling: health claims; soluble dietary fiber from certain foods and coronary heart disease. Final Rule Federal Register Doc. 2004P-0512, filed Dec. 23, 2005.
Friedli, G. L. (1996) Interaction of deamidated soluble wheat protein with other food proteins and metals. PhD Thesis, University of Surrey, Guildford, UK.
German, J. B.; O'Neill, T. E. and Kinsella, J. E. (1985) Film forming and foaming behavior of food proteins. JAOCS 62:1358-1366.
Hamada, J. S. (1992) Effects of heat and proteolysis on deamidation of food proteins using peptidoglutaminase. J. Agric. Food Chem. 40:719-723.
Hamada, J. S. and Marshall, W. E. (1989) Preparation and functional properties of enzymatically deamidated soy proteins. J. Food. Sci. 54:598-601.
Kalra, S. and Jood, S. (2000) Effect of dietary barley β-glucan on cholesterol and lipoprotein fractions in rat. J. Cereal Sci. 31:141-145.
Kato, A. and Nakai, S. (1980) Hydrophobicity determined by a fluorescence probe method and its correlation with surface properties of proteins. Biochim. Biophys. Acta. 624:13-20.
Kawase, S.; Murakami, H.; Matsumura, Y. and Mori, T. (2003) Effects of fragmentation and deamidation on the antioxidative activity of C hordein. J. Oleo Sci. 52:175-184.
Kinsella, J. E. (1976) Functional properties of proteins in foods: a survey. CRC Crit. Rev. Food Sci. Nutr. 7:219-280.
Lan, L.; Zhao, M.; Ren, J.; Zhao, H.; Cui, C. and Hu, X. (2010) Effect of acetic acid deamidation-induced modification on functional and nutritional properties and conformation of wheat gluten. J. Sci. Food Agric. 90:409-417.
Li, D.; Xu, M.; Lundin, L. and Wooster, T. J. (2009) Interfacial properties of deamidated wheat protein in relation to its ability to stabilise oil-in-water emulsions. Food Hydrocoll. 23:2158-2167.
Li, X.; Liu, Y.; Yi, C.; Cheng, Y.; Zhou, S. and Hua, Y. (2010) Microstructure and rheological properties of mixtures of acid-deamidated rice protein and dextran. J. Cereal Sci. 51:7-12.
Liu, G.; Li, J.; Shi, K.; Wang S.; Chen, J.; Liu, Y. and Huang Q. (2009) Composition, secondary structure, and self-assembly of oat protein isolate. J. Agric. Food Chem. 57:4552-4558.
Liu, S.; Low, N. H. and Nickerson M. T. (2009) Effect of pH, salt, and biopolymer ratio on the formation of pea protein isolate-gum arabic complexes. J. Agric. Food Chem. 57:1521-1526.
Malabat, C.; Sanchez-Vioque, R.; Rabiller, C. and Gu, J. (2001) Emulsifying and foaming properties of native and chemically modified peptides from the 2S and 12S proteins of rapeseed (Brassica napus L.). JAOCS. 78:235-242.
Matsudomi, N.; Sasaki, T.; Kato, A. and Kobayashi, K. (1985) Conformational changes and functional properties of acid-modified soy protein. Agric. Biol Chem. 49:1251-1256.
McClements, D. J. (1999) Food emulsions: principles, practice, and techniques. CRC Press: Boca Raton, Fla.
Mejia, C. D.; Mauer, L. J. and Hamaker, B. R. (2007) Similarities and differences in secondary structure of viscoelastic polymers of maize α-zein and wheat gluten proteins. J. Cereal Sci. 45:353-359.
Nakai, S. (1983) Structure-function relationships of food proteins: with an emphasis on the importance of protein hydrophobicity. J. Agric. Food Chem. 31:676-683.
Paraman, I.; Hettiarachchy, N. S. and Schaefer, C. (2007) Glycosylation and deamidation of rice endosperm protein for improved solubility and emulsifying properties. Cereal Chem. 84:593-599.
Pomeranz, Y. and Shands, H. L. (1974) Food uses of barley. Critical Rev. Food Sci. Nutr. 4:377-394.
Shewry, P. R. (1993) Barley seed proteins. In Barley: Chemistry and Technology; MacGregor, A. W. and Bhatty, R. S., eds. American Association of Cereal Chemists Inc.: St. Paul, Minn., USA, pp. 131-197.
Shukla, R. and Cheryan, M. (2001) Zein: the industrial protein from corn. Ind. Crop. Prod. 13:171-192.
Siu, N.; Ma, C.; Mock, W. and Mine, Y. (2002) Functional properties of oat globulin modified by a calcium-independent microbial transglutaminase. J. Agric. Food Chem. 50:2666-2672.
Townsend, A. A. and Nakai, S. (2006) Relationships between hydrophobicity and foaming characteristics of food proteins. J. Food Sci. 48:588-594.
Wang, C.; Tian, Z.; Chen, L.; Temelli, F.; Liu, H. and Wang, Y. (2010) Functionality of barley proteins extracted and fractionated by alkaline and alcohol methods. Cereal Chem. 87(6):597-606.
Wellner, N.; Belton, P. S. and Tatham, A. S. (1996) Fourier transform IR spectroscopic study of hydration-induced structure changes in the solid state of ω-gliadins. Biochem. 1 319:741-747.
Wood, P. J. (2004) Relationships between solution properties of cereal β-glucans and physiological effects—a review. Trend. Food Sci. Technol. 15:313-320.
Yalcin, E.; Celik, S. and Ibanoglu, E. (2008) Foaming properties of barley protein isolates and hydrolysates. Eur. Food Res. Technol. 226:967-974.
Yasumatsu, K.; Sawada, K.; Moritaka, S.; Misaki, M.; Toda, J.; Wada, T. and Ishii, K. (1972) Studies on function properties of food-grade soybean products. Whipping and emulsifying properties of food-grade soybean products. Agr. Biol. Chem. 36:719-727.
Yong, Y. H.; Yamaguchi, S. and Matsumura Y. (2006) Effects of enzymatic deamidation by protein-glutaminase on structure and functional properties of wheat gluten. J. Agric. Food Chem. 54:6034-6040.
Yong, Y. H.; Yamaguchi, S.; Gu, Y. S.; Mori, T. and Matsumura, Y. (2004) Effects of enzymatic deamidation by protein-glutaminase on structure and functional properties of alpha-zein. J. Agric. Food Chem. 52:7094-7100.

1. A method of producing a deamidated protein from barley, comprising the steps of:
a) extracting protein from barley;
b) treating the extracted protein with an alkaline or acidic solution for a sufficient time and temperature for deamidation; and
c) recovering deamidated protein from the solution.

2. The method of claim 1, wherein the deamidated protein is hordein,

3. The method of claim 2, wherein in step (a), hordein is extracted from pearled barley flour with ethanol.

4. The method of claim 3, wherein the ratio of ethanol to flour is 6:1 (v/w),

5. The method of claim 9, wherein the mixture is stirred for about two hours at about 60° C.

6. The method of claim 10, wherein hordein is precipitated and freeze-dried.

7. The method of claim 1, wherein the deamidated protein is glutelin.

8. The method of claim 7, wherein in step (a), pearled barley flour is mixed with an alkaline solution to extract glutelin.

9. The method of claim 8, wherein the ratio of alkaline solution to flour is 10:1 (v/w).

10. The method of claim 9, wherein the mixture is stirred for about 30 minutes at room temperature.

11. The method of claim 7, wherein glutelin is precipitated and freeze-dried.

12. The method of claim 1, wherein in step (b), the protein is deamidated with an alkaline solution.

13. The method of claim 12 wherein the alkaline solution comprises 0.1M to about 0.5M sodium hydroxide.

14. The method of claim 13, wherein in step (b), the reaction temperature is between about 30° C. to about 60° C., and the time of reaction is between about 10 minutes to about 120 minutes.

15. The method of claim 1, wherein in step (c), the deamidated protein is recovered in the form of a freeze-dried protein concentrate.

16. A deamidated glutelin produced by the method of claim 1 and having a degree of deamidation ranging between about 0.5% to about 40%.

17. The glutelin of claim 16, wherein the degree of deamidation ranges between about 1.0% to about 15%.

18. The glutelin of claim 16, wherein the degree of deamidation ranges between about 1.0% to about 2.5%.

19. A deamidated hordein produced by the method of claim 1 and having a degree of deamidation ranging between about 0,7% to about 40%.

20. The hordein of claim 19, wherein the degree of deamidation ranges between about 2.4% to about 4.7%.

21. (canceled)

22. (canceled)
